(19) United States
(12) Patent Application Publication
(10) Pub. No.: US 2023/0324397 A1
(43) Pub. Date: Oct. 12, 2023

(54) Antibody composition for immunotyping of myeloid tumor and use thereof

(71) Applicant: Peking University People's Hospital, Beijing (CN)

(72) Inventors: Yanrong LIU, Beijing (CN); Yazhe WANG, Beijing (CN)

(21) Appl. No.: 18/172,516

(22) Filed: Feb. 22, 2023

(30) Foreign Application Priority Data
Feb. 22, 2022 (CN) 202210159447.8

(51) Int. Cl. G01N 33/574; G01N 33/569; C07K 16/28

(52) U.S. Cl. CPC G01N 33/57492; G01N 33/57426; G01N 33/56972; C07K 16/2866; C07K 16/2896

(57) ABSTRACT

The present disclosure relates to the field of antibody medicine and in particular to an antibody composition for immunotyping of myeloid neoplasms and use thereof, the antibody composition comprising panels of 22-24 antibodies. In the disclosure, combinations of antibodies and corresponding fluorescent labels as well as the method for interpreting the results are optimized, which, with only one tube of 22 or 24 antibodies and one tube of cells at a time, allows for comprehensive and efficient subtyping of acute myeloid leukaemia (AML) and chronic myeloid neoplasms, prediction of CML and part of AML with recurrent genetical abnormalities, and thus a high sensitivity for the diagnosis of myelodysplasia (MDS). It is also possible to identify leukaemia-associated immunophenotypes (LAIP) in this composition that can be used for post-treatment minimal residual disease (MRD) monitoring.

### CROSS-REFERENCE TO RELATED APPLICATION

This patent application claims the benefit and priority of Chinese Patent Application No. 202210159447.8, entitled “ANTIBODY COMPOSITION FOR IMMUNOTYPING OF MYELOID TUMOR AND USE THEREOF” filed on Feb. 22, 2022, the disclosure of which is incorporated by reference herein in its entirety as part of the present application.

### TECHNICAL FIELD

The present disclosure relates to antibody medicament field, in particular to an antibody composition for immunotyping of a myeloid tumor and use thereof.

### BACKGROUND ART

Acute and chronic myeloid neoplasms account for a large part of haematological/lymphatic neoplasms. According to the 2017 World Health Organization (WHO) Classification of Tumors of Haematopoietic and Lymphoid Tissues, acute myeloid neoplasms are classified into acute myeloid leukaemia (AML) and related myeloid precursor neoplasms, which are divided into seven main categories as shown in Table 1. As can be seen from the WHO classification, the diagnosis of haematological/lymphatic neoplasms highlights the importance of morphological/pathological, immunotyping, cytogenetic and molecular (MICI) testing.

In the classification of AML, the first category, AML with recurrent genetic abnormalities, is based on cytogenetics and genes as the final diagnostic basis. However, some of these subtypes have characteristic immunophenotypes that allow a general inference as to which genetically abnormal leukaemia they belong to. The second category, AML with myelodysplasia (MDS)-related changes, requires morphology or pathology to determine MDS haematopoietic dysplasia features; the fifth category, myeloid sarcoma, is a mass that forms outside the bone marrow, consists of mature or immature myeloid cells and requires pathological examination of tissue sections. The fourth category, therapy-related AML, and the sixth category, myeloid proliferations associated with Down syndrome are diagnosed by firstly identifying AML and then combining the identification with the medical history. These categories of AML all require immunotyping to help determine the myeloid origin and acute stage of differentiation, and the final MICM diagnosis is then determined in conjunction with the characteristics described above on the basis of AML diagnosis. The determination of AML is not possible without immunotyping. In addition to these categories, immunotyping plays a key role in the fourth-category, AML not otherwise specified (NOS) and the seventh-category blastic plasmacytoid dendritic cell neoplasm (BPDCN), based on which diagnosis is made.

TABLE 1
2017 WHO classification of
tumors of AML and related precursor neoplasms

1. AML with recurrent genetic abnormalities
AML with t(8;21)(q22;q22); RUNX1-RUNX1T1
AML with inv(16)(p13;q22) or t(16;16)(p13;q11); CBFβ-MYH11
Acute promyelocytic leukaemia (APL) with t(15;17)(q22;q12); PML-RARα
AML with t(9;11)(p22;q23); MLL3-MLL
AML with t(6;9)(p23;q34); DEK-NUP214
AML with inv(3)(q11q26.2) or t(3;3)(q21;q26.2); GATA2.MECOM
AML (megakaryoblastic) with t(1;22) (p13;q13); RBM15-MKL1
Temporary classification: AML with BCR-ABL
AML with mutated NPM1
AML with biallelic mutation of CEBPA
Temporary classification: AML with mutated RUNXI
2. AML with myelodysplasia (MDS)-related changes
3. Therapy-related myeloid neoplasms
4. AML, not otherwise specified (NOS)
AML with minimal differentiation (M0)
AML without maturation (M1)
AML with maturation (M2)
Acute myelomonocytic leukaemia (M4)
Acute monoblastic and monocytic leukaemia (M5)
Pure erythroid leukaemia
Acute megakaryoblastic leukaemia (M7)
Acute basophilic leukaemia
Acute panmyelosis with myelofibrosis
5. Myeloid sarcoma
6. Myeloid proliferations associated with Down syndrome
Transient abnormal myelopoiesis associated with Down syndrome
Myeloid leukaemia associated with Down syndrome
7. Blastic plasmacytoid dendritic cell neoplasm (BPDCN)

In 2017 WHO Classification of Tumors of Haematopoietic and Lymphoid Tissues, chronic myeloid neoplasms are classified into five categories, as shown in Table 2. Generally, immunotyping is not the primary basis for the diagnosis of CMN and is not required for the diagnosis of most chronic myeloid neoplasms. However, it serves to identify the diagnosis.

TABLE 2
2017 WHO classification of chronic myeloid neoplasms

```
1. Myeloproliferative neoplasms (MPN)
      Chronic myeloid leukaemia (CML), BCR-ABL1-positive
      Chronic neutrophilic leukaemia (CNL)
      Polycythaemia vera (PV)
      Primary myelofibrosis (PMF)
            Prefibrotic/early PMF
            Overtly fibrotic PMF
      Essential thrombocythaemia (ET)
      Chronic eosinophilic leukaemia (CEL), NOS
      Myeloproliferative neoplasm, unclassifiable
2. Mastocytosis (3 types)
3. Myeloid/lymphoid neoplasms with eosinophilia and gene
rearrangement (3 types)
4. Myelodysplastic/myeloproliferative neoplasms (MDS/MPN)
      Chronic myelomonocytic leukaemia (CMML)
      Atypical chronic myeloid leukaemia (aCML), BCR-ABL1-negative
      Juvenile myelomonocytic leukaemia (JMML)
      MDS/MPN with ring sideroblasts and thrombocytosis (MDS/
      MPN-RS-T)
      Myelodysplastic/myeloproliferative neoplasm, unclassifiable (UC)
5. Myelodysplastic syndromes (MDS)
      MDS with single lineage dysplasia (MDS-SLD)
      MDS with multilineage dysplasia (MDS-MLD)
      MDS with ring sideroblasts and single lineage dysplasia (MDS-
      RAS-SLD)
      MDS with ring sideroblasts and multilineage dysplasia (MDS-
      RAS-MLD)
      MDS syndrome with excess blasts (MDS-EB)
      MDS with isolated del (5q)
      MDS, unclassifiable (UC)
      Childhood myelodysplastic syndrome
            Refractory cytopenia of childhood (RCC)
```

Several objectives can be achieved by immunotyping of leukaemia/lymphoma using flow cytometry (FCM).

Objective 1: Determination of the presence and type of a tumor. Objective 2: Determination of the subtype. Objective 3: Identification of markers for the next step of minimal residual disease (MRD) assays to search for leukaemia-associated immunophenotypes (LAIP). Objective 4: Detection of expression of markers associated with therapeutic targets. Objective 5: Prediction of genetic abnormalities by detecting markers for certain phenotypes that are highly correlated with specific genetic abnormalities. Examples include AML with t (8; 21) (q22; q22), RUNX1-RUNX1T1, and APL with t(15; 17)(q22; q12), PML-RARα. It is important to immunotype APL, because such patients have a high mortality rate in the early stage of the disease and need to be given retinoic acid or arsenic-based chemotherapy as soon as possible to save their lives. The long-term efficacy is excellent after the patient has survived the early risk phase. Early diagnosis is therefore crucial. Immunotyping and morphological determination are the fastest methods of leukaemia diagnosis available compared with gene detection and genetic testing, which take at least 2-3 weeks, a time longer than it takes for immunotyping. Therefore, w % ben both immunotyping and morphological testing suggest APL with t(15; 17)(q22; q12). PML-RARα, immediate clinical initiation of targeted chemotherapy is needed to reduce early mortality.

At present, the clinical assays described above require dozens of antibodies. The four-color antibody staining panel for immunotyping of acute leukaemia was described in the Chinese Journal of Haematology in 2015, where for AML typing a total of 10 tubes of 38 antibodies are required for the assay. The consensus on the four-color antibody staining panel is only for acute leukaemia but not for chronic myeloid neoplasms, particularly, e.g. MDS. 2012 Euroflow Europe published an 8-color antibody staining panel for immunotyping of haematological neoplasms. The antibody panel used for further analysis of AML/MDS after the initial detection with screening tubes is shown in Table 3, where 7-tube-8-color antibody panel with 4 common antibodies per tube was used. A total of 56 antibodies were tested and 28 repetitive antibodies were used, leaving 32 valid antibodies.

TABLE 3
Antibody panel used for AML/MDS by Euroflow ®

| Pacific | Pacific | | | | | | |
|---|---|---|---|---|---|---|---|
| Blue | Orange | FITC | PE | PerCP | PE-Cy7 | APC | APC-H7 |
| HLA-DR | CD45 | CD16 | CD13 | CD34 | CD117 | CD11B | CD10 |
| HLA-DR | CD45 | CD35 | CD64 | CD34 | CD117 | CD300e | CD14 |
| HLA-DR | CD45 | CD36 | CD105 | CD34 | CD117 | CD33 | CD71 |
| HLA-DR CD45 | TDT | CD56 | CD34 | CD117 | CD7 | CD19 | |
| HLA-DR CD45 | CD15 | NG2 | CD34 | CD117 | CD22 | CD38 | |
| HLA-DR CD45 | CD42/61 | CD203c | CD34 | CD117 | CD123 | CD4 | |
| HLA-DR CD45 | CD41 | CD25 | CD34 | CD117 | CD42B | CD9 | |

Currently available antibody panels are costly due to the repeated use of various gated antibodies, resulting in the use of more total antibodies. More tubes are required and it is difficult to accurately analyze the relationship between the antibodies detected in different tubes, which affects the determination of cell lineage, stage of differentiation and discrimination between benign and malignant cells. There is therefore an urgent need to design an antibody composition that can simultaneously perform comprehensive immunotyping, subtyping, MRD marker and therapeutic target screening, and predictive genotyping of acute and chronic myeloid neoplasms. There are many issues to be addressed by those skilled in the art to design the antibody composition described above, including the selection and combination of antibodies and fluoresceins, and the degree of expertise as well as clinical experience required for those skilled in the art.

In addition, the diagnosis of monocytic leukaemia (AML-M4/5) by immunotyping is often very difficult mainly due to the difficulties in identifying immature granulocytes, abnormal monocytes and promonocytes in the specimen. The differentiation between promonocytes and immature granulocytes determines whether the patient is diagnosed with AML-M2 or AML-M4/5. Secondly, in patients with mononucleosis, whether the monocytes are mature or immature determines whether the patient has CMML or AML-M4. The identification and diagnosis are important as the treatment varies between diseases. Furthermore, in some specimens of MDS, due to granulocyte degranulation, mature granulocytes are difficult to distinguish from monocytes in the CD45/SSC (side scatter) plot, where the SSC intensity is reduced and located in the place of monocytes, seriously affecting their identification. Therefore, there is also an urgent need for a method for identifying promonocytes to solve the above technical problems.

### SUMMARY

To solve the above problems, provided in the present disclosure is an antibody composition for immunotyping of acute myeloid leukaemia and related precursor neoplasms (together referred to as AML) as well as chronic myeloid neoplasms, specifically including panels of 22 or 24 antibodies. The composition is primarily used for subtyping acute and chronic myeloid neoplasms, screening for MRD markers and therapeutic targets and predicting genotypes, so as to achieve comprehensive immunotyping of myeloid neoplasms.

A panel of 19 antibodies in one tube as described in Application No. CN2021110670743 is used for primary screening of haematological neoplasms (first step of the screen). The haematological neoplasms are classified into 9 categories: AML, T-lineage acute lymphoblastic leukaemia (ALL-T), B-lineage acute lymphoblastic leukaemia (ALL-B), mixed phenotype acute leukaemia (MPAL), B-cell non-hodgkin lymphomas (NHL-B), T-cell non-hodgkin lymphomas (NHL-T), NK-cell non-hodgkin lymphomas (NHL-NK), plasma cell neoplasm (PCN) and chronic myeloid neoplasm, and 7 major categories of neoplasm, AML, ALL-T, ALL-B, MPAL, NHL-B, NHL-T and PCN are clearly diagnosed. Combined with the 22-24 antibodies in one tube for myeloid neoplasm diagnosis (second step of the test) in this application, a total of 2 tubes of antibodies allows for comprehensive immunotyping, subtyping, MRD marker and therapeutic target screening, and predictive genotyping of haematological neoplasms.

To achieve the above objectives, the present disclosure provides the following technical solutions.

The first aspect of the present disclosure provides three panels of antibody composition: a first panel of antibodies including 24 antibodies with 23 colors for immunotyping of myeloid neoplasms, including AML and chronic myeloid neoplasms; a second panel of antibodies including 22 antibodies with 21 colors for immunotyping of AML; and a third panel of antibodies including 22 antibodies with 22 colors for immunotyping of chronic myeloid neoplasms.

In some embodiments, antibody species and fluoresceins in combination therewith of the three panels of antibodies are shown in Table 4.

TABLE 4
Antibody and fluorescein combinations for myeloid neoplasms.

| | Myeloid | | Chronic |
|---|---|---|---|
| Fluorescein | neoplasm | AML | myeloid neoplasm |
| BV421 | CXCR4 | CXCR4 | CXCR4 |
| SB436 | CD105 | | CD105 |
| eFluor450 | CD14 | CD14 | CD14 |
| BV510 | CD45 | CD45 | CD45 |
| BV570 | CD16 | CD16 | CD16 |
| BV605 | HLA-DR | HLA-DR | HLA-DR |
| BV650 | CD33 | CD33 | CD33 |
| BV711 | CD10 | | CD10 |
| BV750 | CD4 | CD4 | CD4 |
| BV785 | CD123 | CD123 | CD123 |
| BB515 | CD11b | CD11b | CD11b |
| FITC | CD61 and | CD61 and | |
| | CD41 | CD41 | |
| cFluor B548 | CD15 | CD15 | CD15 |
| PE | CD13 | CD13 | CD13 |
| PE-Dazzle594 | CD71 | CD71 | CD71 |
| PE-Cy5 | CD117 | CD117 | CD117 |
| PerCP-Cy5.5 | CD34 | CD34 | CD34 |
| PerCP-eF710 | CD9 | CD9 | CD38 |
| PE-Cy7 | CD11c | CD11c | CD11c |
| APC | CD300e | CD300e | CD300e |
| Alexa Fluor700 | CD64 | CD64 | CD64 |
| APC-Fire750 | CD36 | CD36 | CD36 |
| APC-Fire810 | CD25 | CD25 | CD25 |
| Total | 24 | 22 | 22 |

In some embodiments, the antibodies are monoclonal antibodies.

The second aspect of the present disclosure provides a kit for immunotyping of a myeloid neoplasm, including any panel of antibodies described above.

In some embodiments, the kit further includes an erythrocyte lysing solution and a buffer.

The third aspect of the present disclosure provides a system for detecting an immunophenotype of a myeloid neoplasm, including a test moiety and an analyzing moiety, where

the test moiety is used to attain test results of a sample using a tube of agent for testing the sample with flow cytometry, where the agent includes any panel of antibodies described above;

the analyzing moiety is used to analyze the test results from the test moiety to subtype myeloid neoplasms, identify LAIP markers for MRD testing, screen for therapeutic targets, and predict genotypes.

In some embodiments, when the system is used for detecting immunophenotypes of AML and/or CMN, steps of detection include:

preparing a flow cytometry onboard sample after processing a sample to be tested using the panel of antibodies; performing a flow cytometry onboard assay; where

gating in the flow cytometry onboard assay is as follows:

gating live cells as R1, removing debris and dead cells, and gating lymphocytes, granulocytes, monocytes, immature cells, and erythroblasts within R1 gate using CD45/SSC;

analyzing antigen expression within different cell gates, including:

analyzing AML, which includes immunotyping of myeloid immature cells, granulocytes and monocytes; and

analyzing chronic myeloid neoplasms, which includes immunotyping of immature myeloid cells, granulocytes, monocytes and erythroblasts.

In some embodiments, the immunotyping of granulocytes and monocytes includes distinguishing promonocytes from mature monocytes and/or distinguishing promonocytes from immature granulocytes using two-dimensional CXCR4/CD36 plot. In some embodiments, the expression of CXCR4 in promonocytes is higher than that in mature monocytes and granulocytes, and CD36 is distributed from negative to positive.

Specifically, maturity of monocytes is analyzed using monocyte-related markers included in the antibody composition of the present disclosure, e.g. CXCR4, CD33, CD64, CD14, CD300e, CD36, HLA-DR. CD15. CD11c, CD4, CD45 and SSC.

The fourth aspect of the present disclosure provides use of the antibody composition, the kit or the system in the preparation of a product for myeloid neoplasm diagnosis, therapeutic target screening, and screening of monitoring marker for MRD.

The myeloid neoplasms in the present disclosure include: AML and related precursor neoplasms, and chronic myeloid neoplasms.

In further embodiments, the AML and related precursor neoplasms mainly include 7 categories: 1. AML with recurrent genetic abnormalities; 2. AML with MDS-related changes: 3, therapy-related myeloid neoplasms; 4. AML, not otherwise specified (NOS); 5, myeloid sarcoma; 6, myeloid proliferations associated with Down syndrome, and 7. blastic plasmacytoid dendritic cell neoplasm (BPDCN).

The antibody compositions described herein are used for screening of MRD markers in these 7 disease categories. The first and fourth categories of AML subtypes are immunotyped, and genotypes of part of AML with recurrent genetic abnormalities are predicted.

In further embodiments, the chronic myeloid neoplasms include: myeloproliferative neoplasms (MPN), mastocytosis; myeloid/lymphoid neoplasms with eosinophilia and gene rearrangement, myelodysplastic/myeloproliferative neoplasms (MDS/MPN), and myelodysplastic syndromes (MDS).

After initial screening in the first step, patients with haematological neoplasms are excluded from having AML and identified to have chronic myeloid neoplasms, which are then subtyped (Table 2). In patients with clinically suspected MPN and MDS, three conclusions can be made, i.e., supportive diagnosis, suspicious diagnosis and unsupportive diagnosis. For CMML and JMML, the presence of mononucleosis and predominant presence of mature or promonocytes can aid the diagnosis. For the screening of MRD makers, immunotyping is not used for most chronic myeloid neoplasms, but for MDS, as in AML. Screening for therapeutic targets and genetic phenotypes is mainly conducted on patients positive for CML BCR-ABL, where tyrosine kinase inhibitors can be used. Although immunotyping is not a definitive diagnosis basis for this disease, some diseases have typical immunophenotypic features that are suggestive of a diagnosis.

The fifth aspect of the present disclosure provides a method for identifying promonocytes from mature monocytes and/or promonocytes from immatue granulocytes, including detecting the CXCR4/CD36 expression of cell membrane in a sample to be tested for analysis of promonocytes.

In some embodiments, the method further includes detecting one or more of CD33, CD64, CD14, CD300e, HLA-DR, CD15, CD10, CD11b, CD11c, CD4, CD16, CD45 and SSC intensity as other monocyte markers.

In a specific embodiment, the expression of CXCR4 is higher in promonocytes than in mature monocytes and granulocytes, while CD36 is distributed from negative to positive. In combination with other monocyte-related markers in the antibody compositions of the present disclosure, e.g. CD64+CD14−, CD11c+/−CD4+, CD33st+CD15− and CD14−CD300e−, as well as the location below monocyte R5 in a CD45/SSC plot, the cells are identified as promonocytes, so that CXCR4+CD36dim+/− promonocytes. CXCR4−CD36− promyelocytes and CXCR4+(weaker than promonocytes) CD36st+ mature monocytes are distinguished.

Based on the above technical solutions, it is obvious that the embodiments of the present disclosure have the following beneficial effects.

Panels of 22-24 antibodies are utilized for immunotyping of myeloid neoplasms. When a primary screening tube for haematological neoplasms is used in conjunction, only 2 tubes of antibody combination are required for the comprehensive immunotyping of haematological neoplasms. Currently, it is common to perform conventional flow cytometric immunotyping of AML, CML and MDS using 8-10 colors staining antibody panels, each disease requiring 4-5 tubes of antibody panels (36-40 antibodies), whereas only 1 tube of antibody composition (22-24 antibodies) is used in the present disclosure to subtype acute or chronic myeloid neoplasms, which therefore reduces the requirement for specimen volume and operation procedures, reduces labor intensity and saves operation time. The present disclosure reduces the repeated application of gated antibodies, and increases the number of effective antibodies used. Meanwhile, it allows for observation of the simultaneous expression of 22-24 antibodies and analysis of the relationship between phenotypes and antibody panels, increasing the accuracy, specificity and sensitivity of the diagnosis of myeloid neoplasms. Also disclosed in the present disclosure are methods of analysis for identifying promonocytes from mature monocytes and immature granulocytes using a two-dimensional CXCR4/CD36 plot; the above methods are used to identify AMLM4/5 from other AML and from CMML. In MDS, granulocytes are often difficult to be distinguished from monocytes when granularity decreases, and the method of the present disclosure can also be used to distinguish monocytes from abnormal granulocytes.

### BRIEF DESCRIPTION OF THE DRAWINGS

FIGS. 1A-1B illustrate a method for gating promonocytes using CXCR4/CD36. FIG. 1A shows the results of a patient with AML-M5; and FIG. 1B shows results of a patient with CMML.

FIG. 2 illustrates a method of gating for MDS. A specimen of normal marrow is shown.

FIGS. 3A-3B show the detection results of an AML-M2 case. FIG. 3A shows the detection results of an AML-M2 case using the antibody composition of the present disclosure, and FIG. 3B shows the detection results of an AML-M2 case by preliminary screening.

FIGS. 4A-4B show the detection results of an AML-M5 case. FIG. 4A shows the detection results of an AML-M5 case using the antibody composition of the present disclosure, and FIG. 3B shows the detection results of an AML-M5 case by preliminary screening

FIGS. 5A-5B show the detection results of AML with basophilic differentiation.

FIG. 5A shows the detection results of AML with basophilic differentiation using the antibody composition of the present disclosure, and FIG. 5B shows the detection results of AML with basophilic differentiation by preliminary screening.

FIGS. 6A-6B show a case of AML with mutated NPM1. FIG. 6A shows the results of a case of AML with mutated NPM1 using the antibody composition of the present disclosure, and FIG. 6B shows the results of a case of AML with mutated NPM1 by preliminary screening.

FIGS. 7A-7B show the detection results of a case of AML-M2 with t(8; 21). FIG. 7A shows the detection results of a case of AML-M2 with t(8; 21) using the antibody composition of the present disclosure, and FIG. 7B shows the detection results of a case of AML-M2 with t(8; 21) by preliminary screening.

FIGS. 8A-8B show the detection results of a case of APL with t(15; 17). FIG. 8A shows the detection results of a case of APL with t(15; 17) using the antibody composition of the present disclosure, and FIG. 8B shows the detection results of a case of APL with t(15:17) by preliminary screening.

FIGS. 9A-9B show the detection results of a CMML case. FIG. 9A shows the detection results of a CMML case using the antibody composition of the present disclosure, and FIG. 9B shows detection results of a CMML case by preliminary screening.

FIG. 10 shows the detection results of an MDS case.

FIG. 11 shows the detection results of a CML case.

FIG. 12 shows the detection results of an MPN-ET case.

FIG. 13 shows the detection results of an MDS/MPN UC case.

Abbreviations in the figures are as follows: Mo-monocytes, MM-mature monocytes, PMC-promonocytes, Ly-lymphocytes, EC-erythrocytes, GC-granulocytes, IMC-immature myeloid cells, Eo-eosinophils, Bo-basophils.

### DETAILED DESCRIPTION OF THE EMBODIMENTS

The following examples are intended to illustrate the disclosure, not to limit the scope of the disclosure. Unless specifically indicated, the technical means used in the examples are conventional means known to those skilled in the art. The experimental methods used in the following examples are conventional if not otherwise specified. All materials, reagents etc. used in the following examples are commercially available, if not otherwise specified.

In the present disclosure, flow cytometry is used to immunotype specimens of bone marrow fluid, thoraco-abdominal fluid and peripheral blood from clinical patients, and specimens identified as possible AML and chronic myeloid neoplasms by primary screening is subjected to a second step of comprehensive immunotyping. The panel of 19 antibodies used in the primary screening is described in CN patent application No. 2021110670743.

### Example 1. Preparation of Regents

Three panels of antibodies were used for the diseases.

The first panel of 24 antibodies with 23 colors of fluoresceins was prepared according to the antibody combination for myeloid neoplasms in Table 4. The antibodies and the fluoresceins were mixed accordingly, put into a container, and used for immunotyping of all specimens identified as myeloid neoplasms (AML and chronic myeloid neoplasms).

The second panel of 22 antibodies with 21 colors of fluoresceins was prepared according to the antibody combination for AML in Table 4. Components were the same as those in the myeloid group except that anti-CD105-SB436 and anti-CD10-BV711 were not added. The antibodies and the fluoresceins were mixed accordingly, put into a container, and used for immunotyping of specimens identified as AML.

The third panel of 22 antibodies with 22 colors of fluoresceins was prepared according to the antibody combination for chronic myeloid neoplasms in Table 4. Components were the same as those in the myeloid group except that anti-CD41-FITC and anti-CD61-FITC were not added and anti-CD9-PerCP-eF710 was replaced with anti-CD38-PerCP-eF710. The antibodies and the fluoresceins were mixed accordingly, put into a container, and used for immunotyping of specimens identified as chronic myeloid neoplasms.

The antibodies above are commercially available, and the antibodies used in the examples were purchased from BD Biosciences, Biolegend, Beckman Coulter, and Thermo Fisher Scientific.

The above three panels of antibodies were prepared into a kit for detecting myeloid disease, AML and chronic myeloid disease. The kit further included an erythrocyte lysing buffer and phosphate buffer saline (PBS). The erythrocyte lysing buffer was either self-made or purchased from a company (BD Biosciences, for example).

### Example 2. Analysis of Immunophenotypes of Myeloid Neoplasms by Flow Cytometry with Panels of 22-24 Antibodies

1. Experimental Materials and Instrument

1. Materials: 10×PBS, and a BD fluorescence activating cell sorter (FACS) Lysing Solution dedicated for flow cytometer.

2. Instruments: CytekNL-3000 full spectrum flow cytometer, provided with 3 lasers (405 nanometers (nm), 488 nm and 635 nm) and 38 fluorescence detectors; a desktop low-speed centrifuge, and a vortex mixer.

II. Methods

1. Sample Collection

1-3 mL of human bone marrow fluid was sampled and immediately placed into a heparin anticoagulation tube, which was then quickly inverted several times to prevent coagulation of the sample. Chest and abdominal fluid, lavage fluid and other cells were sent to the laboratory immediately after collection, and stored in a refrigerator at 4° C. Flow cytometry (FCM) assay was completed within 48 hours, as per instructions.

2. Sample Preparation.

(1) Cell counting: 10 μl of bone marrow fluid was taken, 150 μl of PBS was added and mixed well, the number of cells per microliter (μl) was counted using Myriad FCM, the cell concentration was adjusted to 5-10×10⁶/100 μl according to the results, and 50 μl-100 μl of cells were added to a flow tube.

(2) Antigen Staining.

a) The corresponding fluorescein-labeled monoclonal antibody premix according to Table 4 and bone marrow samples were added to each tube separately, mixed thoroughly, incubated for 15 min at room temperature and protected from light.

b) Haemolysis: 2 ml of 1×FACS lysing solution was added, vortexed at low speed to mix well with the samples, allowed to stand for 8-10 min at room temperature and protected from light. Centrifugation at 300 g for 5 min and washing were carried out and a resulting supernatant was discarded.

c) Washing: 1 ml of PBS containing 0.1% NaN₃ and 1-2% BSA were added for washing by centrifugation at 300 g for 5 min, and a resulting supernatant was discarded. 200 μl of PBS was added to re-suspend the cells for further onboard assay.

(3) Onboard Assay.

a) Determination of the optimum voltage and compensation: the voltage was set according to the routine operation of the spectral flow cytometer and single-stained samples were prepared with reference to the fluorescent color combination of the kit for instrument setting.

b) Instrument set-up, calibration and quality control (QC): CytekNL-3000 was switched on to warm up the machine for more than 20 min and rinsed with deionized water, and the internal QC was tested to ensure that the values were within the normal range. AL-PANAL© was called for sample loading to collect data.

c) On-board assay: 50-100,000 cells per tube were obtained according to the instrument parameters. When the assay could not be run on time, 0.5 ml of 1% paraformaldehyde was added, mixed well with the cells, and stored in a refrigerator at 4° C., and the assay was conducted within 24 hours.

III. Data Analysis Using Kluzaa© Software.

(i). Analysis on AML

1. Debris, adherent cells and dead cells were removed using forward scatter (FSC)/SSC, and single live cells were gated as R1.

2. R1 gate cells were shown, and a CD45/SSC plot was constructed. According to the distribution of CD45 and SSC, lymphocytes, monocytes, granulocytes, erythroblasts and immature cells were gated and various colors were assigned. Lymphocytes (R1) had the highest CD45 expression and the lowest SSC intensity; monocytes (R5) had lower CD45 expression than lymphocytes, higher SSC intensity than lymphocytes but lower SSC intensity than granulocytes; granulocytes (R4) had lower CD45 expression than monocytes and the highest SSC intensity: erythroblasts (R6) showed negative CD45 expression and the same SSC intensity as lymphocytes; immature cells (R3) had lower CD45 expression than lymphocytes and lower or equal SSC intensity than/to lymphocytes. In normal bone marrows, the normal proportions of cell populations are as follows: lymphocytes 20% to 40%, monoyctes 2% to 8%, erythroblasts 2% to 15%, and immature cells less than 5%. It was observed whether the proportion of each population was normal and whether the proportion of immature cells increased.

A series of two-dimensional dot plots with 2 antibodies was constructed, mainly including CD34/CD117, CD33/CD117, HLA-DR/CD123, CD13/CD11B, CD13/CD16, CD15, HLA-DR/CD11B, CD14/CD16, CD14/CD16 CD14/CD300E, CD36/CXCR4, CD64/CD15, CD64/CD14, CD33/CD15, CD11C/CD4, etc. First, R1 gate cells were observed for the expression of antigens in these plots. Given that different cell populations were marked with different colors, the antigen expression in different cell populations could be analyzed. Among the specimens of some myeloid neoplasms, the boundary of immature cells in the CD45/SSC plot was unclear. Herein, CD34+CD117+ or CD34−CD117+ cells were gated using CD34/CD117 plots so that the immature myeloid cells could be gated and analyzed better.

4. The expression of the antigens described in the present disclosure was analyzed mainly in immature cells, granulocytes and monocyte gates. The function of the antigens and their expression on normal blood cells are shown in Table 5. The criteria for subtyping AML using the 22-24 antibodies in the present disclosure are shown in Table 6.

TABLE 5
Function of the antigens and their expression on normal cells

| Antigen | Function/expressing cells |
|---|---|
| CXCR4 | Promonocytes, eosinophils and basophils (strong st+); |
| | distinguishment between granulocytes and promonocytes. |
| CD105 | Immature erythrocytes |
| CD14 | Immature and mature monocytes |
| CD45 | Typing leukocytes; immature cells (often weakly expressed, |
| | dim+) |
| CD16 | Stab granulocytes, segmented granulocytes, atypical |
| | monocytes, natural killer (NK) cells, and dendritic cells (DC); |
| HLA-DR | Promyelocytes and mature monocytes |
| CD33 | A myeloid marker; myeloid precursors, monocytes (stronger |
| | expression than granulocytes), granulocytes, and mast cells |
| CD10 | Segmented granulocytes, precursor B and T cells |
| CD4 | T-cell subpopulation, monocytes and plasmacytoid dendritic |
| | cells (PDC) |
| CD123 | Basophils, PDC, precursor cells, and megakaryocytes |
| CD11b | Granulocytes (appears in myelocyte), basophils, monocytes, |
| | NK cells, T- and B-cell subpopulations, and DC |
| CD61/ | Megakaryocytes and platelets |
| CD41 | |
| CD15 | Granulocytes (appears in promyelocytes), monocytes, and |
| | eosinophils |
| CD13 | Granulocytes, monocytes, myeloid progenitors, endothelial |
| | cells, epithelial cells, and DC |
| CD71 | Erythroblasts and proliferating cells, including |
| | megakaryocytes |
| CD117 | Immature myeloid cells, mast cells (strong expression), |
| | activated NK cells, and basophils (weak expression) |
| CD34 | T-cells, B-cells, immature myeloid cells, hematopoietic stem |
| | cells (HSC), and endothelial cells. |
| CD38 | Immature myeloid cells and mature monocytes, and |
| | plasmacytes (strong expression) |
| CD9 | Eosinophilic and basophils (strong expression), mast cells, |
| | platelets/megakaryocytes, Pre-B cells; |
| CD11c | Mature granulocytes, eosinophilic and basophils, monocytes, |
| | NK cells, T/B subpopulations, and DC |
| CD300e | Mature monocytes |
| CD64 | Granulocytes (appears in promyelocytes and strongly |
| | expressed, deceased expression in mature granulocytes) and |
| | monocytes (strong expression) |
| CD36 | Promonocytes (weak expression), mature monocytes (strong |
| | expression), erythrocyte series, and platelets/megakaryocytes |
| CD25 | Activated T/B cells, monocytes, DC subpopulation, regulative |
| | T cells (Treg) |

CXCR4/CD36 was used to differentiate promonocytes from immature granulocytes and mature monocytes.

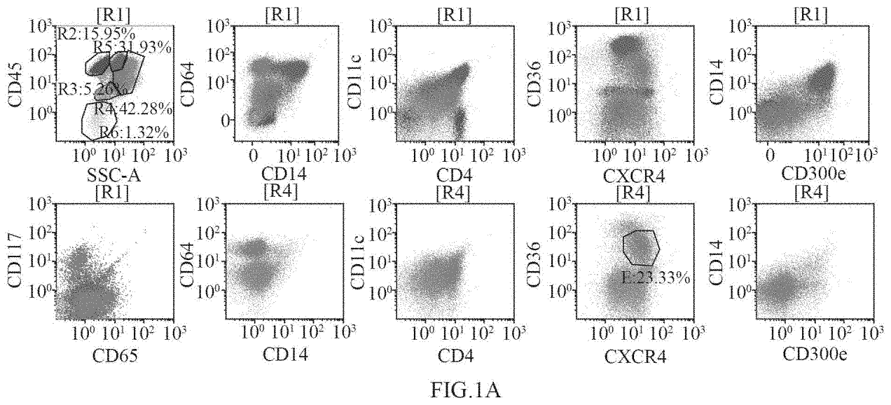

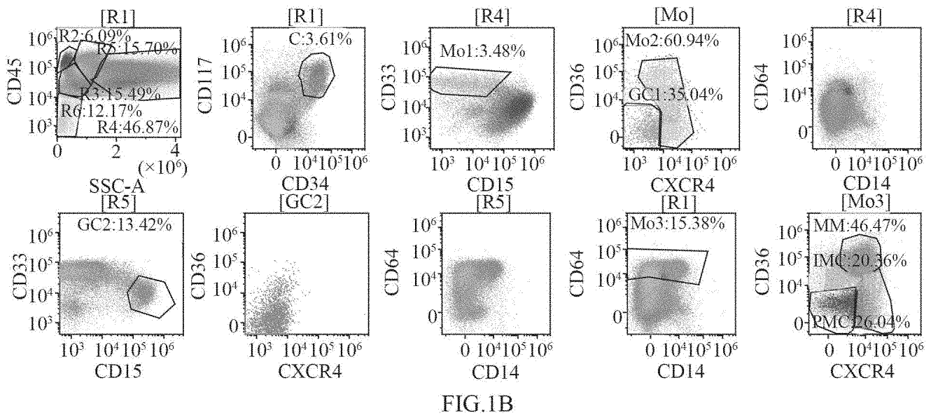

In some AML or MDS specimens, conventional gating methods such as CD45/SSC and CD33/CD15 or CD64/CD14 do not effectively gate monocytes and granulocytes, and promonocytes are often included in the granulocyte gate. Therefore, the present disclosure adopted CXCR4/CD36 to analyze promonocytes, and CXCR4 expression was stronger in promonocytes than in mature monocytes and granulocytes, while CD36 was distributed from negative to positive. In combination with other monocyte-associated markers in the panels, such as CD64+CD14−, CD11c+/−CD4+, CD33st+CD15−, CD14−CD300e−, and the location below monocyte R5 in the CD45/SSC plot, promonocytes were identified. Promonocytes were identified from immature granulocytes (FIG. 1).

Promyelocytes had the phenotype of CD64st+CD33st+CD14−CD300e−CD11c−CD4−, but did not express CXCR4. They were located at the lower edge of the R4 gate in the CD45/SSC plot and had higher SSC intensity than mature granulocytes. In some CMML specimens, granulocytes and monocytes were overlapped with each other and neither CD45/SSC nor CD64/CD14 could effectively separate monocytes from promyelocytes, so a CD36/CXCR4 plot was used in the present disclosure to identify CXCR4−CD36− promyelocytes from CXCR4+CD36dim+/− promonocytes. Thus, AML-M2 versus AML-M4, and AML-M5 versus CMML were distinguished.

TABLE 6
Characteristics of AML subtypes

| AML | % immature | | |
|---|---|---|---|
| subtype | granulocytes | % promonocytes | Phenotype |
| 1. AML-M0 | ≥20% | — | Immature granulocytes have a low SSC |
| | | | intensity similar to lymphocytes. At least |
| | | | one of myeloid markers, e.g. CD33, |
| | | | CD13, CD117, CD15, and MPO, is |
| | | | expressed. |
| 2. AML-M1 | ≥90% | — | Immature granulocytes have higher SSC |
| | | | intensity than lymphocytes. CD34 and/or |
| | | | CD117, as well as CD38, HLA-DR, |
| | | | CD33, CD13, and CD15 are expressed. |
| 3. AML-M2 | ≥20% | — | Immature granulocytes have higher SSC |
| | | | intensity than lymphocytes. CD34 and/or |
| | | | CD117 are generally expressed, and |
| | | | CD38, HLA-DR, CD33, CD13, and |
| | | | CD15 are mostly expressed as well. |
| 4. AML-M4 | ≥20% | ≥20% | There is an increased proportion of |
| | | | monocytes. Promonocytes accounts for |
| | | | the most. The cells are CD64st+CD33st+ |
| | | | CD14+/-CD300e−CXCR4+, and weak |
| | | | CD36+, with variable expression of |
| | | | CD117 and CD34. Immature |
| | | | granulocytes are also present, and have |
| | | | a similar phenotype to AML-M2 |
| 5. AML-M5 | — | ≥20% | Promonocytes has the same phenotype as |
| | | | AML-M4. |
| 6. Pure erythroid | — | — | There are more than 80% of erythrocytes |
| leukaemia | | | series, and 30% of proerythroblasts. The |
| | | | phenotype is D71+CD36+CD235a+, |
| | | | with the erythroblasts >80%, and unstable |
| | | | CD117, CD105 and CD33 expression. |
| 7. AML-M7 | >20%, | — | Blast cells account for more than 20%. |
| | | | CD117, CD33, and CD34 are expressed, |
| | | | but CD34− or CD33− often occurs. More |
| | | | than one of megakaryocyte markers CD41, |
| | | | CD61, CD36, CD9 are expressed and |
| | | | CD71 is also expressed. |
| 8. AML with | | | |
| recurrent genetic | | | |
| abnormalities | | | |
| (1) APL with | ≥20% | — | Immature granulocytes have high SSC |
| t(15;17) | | | intensity similar to granulocytes, often |
| | | | similar to mature granulocytes. CD117, |
| | | | CD38, CD9, CD123, CD33, CD13 are |
| | | | expressed and CD64 is weakly epressed. |
| | | | Most cells do not express CD34 and |
| | | | HLA-DR |
| (2) AML with | variable | — | The phenotype is mostly similar to AML- |
| t(8;21) | proportions | | M2, but about 60% of immature cells |
| | | | express CD19, CD56 and weak CD33. |
| | | | The cells also express CD34, CD117, |
| | | | CD38, and HLA-DR |
| (3) AML with | ≥20% | ≥20% | AML-M4 with eosinophilia. |
| inv(16) | | | |
| (4) AML with | ≥20% | — | SSC intensity is lower than that in APL |
| mutated NPM1 | | | cells. CD34 and HLA-DR are usually not |
| | | | expressed, CD33 is strongly expressed, |
| | | | and CD64 is not expressed, Belonging to |
| | | | AML-M2 |
| (5) AML with | — | ≥20% | Promonocytes express CD117, but often |
| mutated NPM1 | | | not express CD34. Some specimens are |
| | | | positive for HLA-DR, express CD33st, |
| | | | CD64st, CXCR4, CD11c, and some |
| | | | express CD36, CD11b, and CD15, |
| | | | belonging to AML-M5. |

5. Determination of LAIP Markers.

Antigens that are aberrantly expressed on leukemic cells are called LAIP. LAIP is one of the important characteristics that distinguishes leukemic cells from normal haematopoietic cells and is the basis for FCM detection of MRD. The antibody panels of the present disclosure were used to screen for abnormal immunophenotypes in patients with AML and chronic myeloid neoplasms for MRD monitoring.

The LAIP described primarily includes:

(1) cross-series antigen expression or cross-lineage antigen expression, e.g. in the case where AML cells express lymphoblastic antigens: CD19, CDT CD56, CD5, etc. (2) Synchronous expression of early and late antigens. Under normal circumstances, the expression of antigens in different series of cells at different stages of differentiation is strictly controlled by genes, and CD34, CD15, or CD11b are not expressed at the same time. The antigens are expressed in sequence. However, leukaemia cells do not express in the normal order. Therefore, CD34+CD15+ or CD1b+ is LAIP. (3) Abnormal antigen expression. Normally, cellular antigen expression intensity of each series at each stage is consistent. AML cells often show enhanced or reduced expression, or even no expression of antigens such as CD34, CD117, CD33, CD13, CD38, and HLA-DR.

Each of the 25 antigens included in this disclosure was analyzed individually to determine if they were LAIP markers.

6. Screening of markers related to therapeutic targets. There are more than 10 targeted drugs for AML, among which the only one targeting the cell surface antigen phenotype is Gituzumab Ozogamicin, which consists of a chemotherapeutic drug coupled to a monoclonal antibody (artificial immune protein) targeting the CD33 protein. The disclosure therefore provided information on CD33 expression abnormalities, thereby providing guidance for clinical administration.

7. Prediction of genotypes. Certain phenotypes are highly correlated with specific genetic abnormalities, and the detection of these markers can predict whether genetic abnormalities are present or not. Several types of AML with recurrent genetic abnormalities with typical phenotypic features are listed in Table 6, where AML with t(8:21)(q22; q22), RUNX1-RUNX1T1 tends to be manifested morphologically and immunophenotypically as AML-M2, and immunophenotypically immature myeloid cells tend to express CD34, CD117. CD38 and HLA-DR. CD33 is typically weakly expressed and about 60% abnormally express the B-lineage-associated marker CD19 and the NK-associated marker CD56. If there are typical immunophenotypic features, there is more than 90% correlation with AML with t(8; 21)(q22; q22), RUNX1-RUNX1T1 genotype leukaemia. For APL with t(15; 17)(q22; q12), PML-RARα genotype leukaemia, the typical patient's bone marrow morphology tends to show a marked increase in promyelocytes and more granules in cells; the immunotyping shows a marked increase in the proportion of CD117+ immature cells, and high side scatter (SSC) intensity of cells, with the cells characterized by the absence of CD34 and HLA-DR expression, strong expression of CD33, and simultaneous expression of CD9, CD123 and CD64 (weak). This phenotype is often indicative of APL with t(15:17)(q22; q12), PML-RARα genotype leukaemia. There's high similarity between AML with mutated NPM1 and APL, while they were distinguished by the low SSC intensity and weak MP0 expression.

(ii) MDS Analysis.

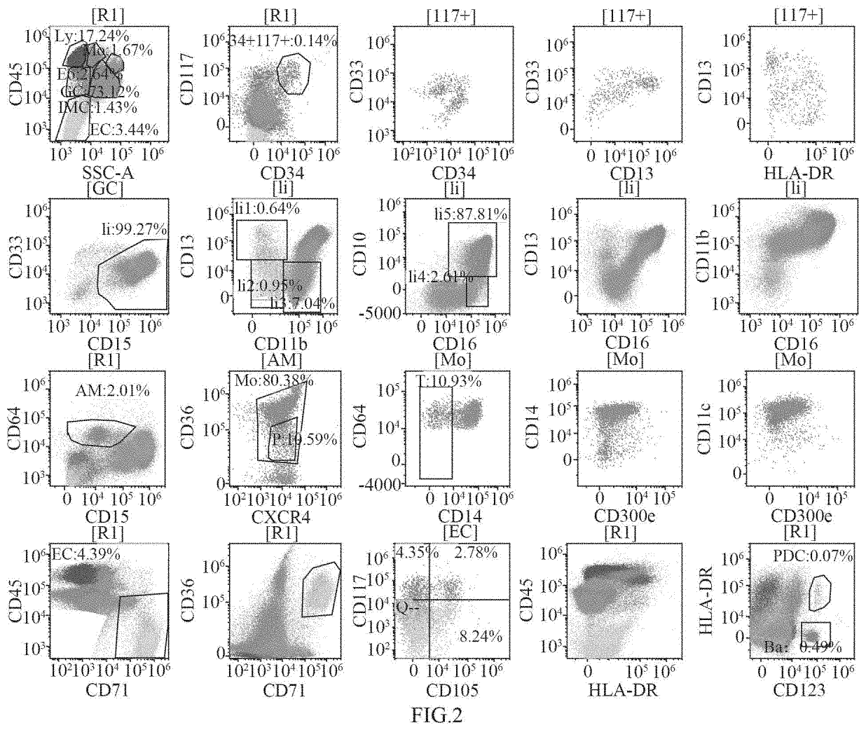

MDS belongs to chronic myeloid neoplasms and is listed separately herein because of the complexity of analysis. The gating method is shown in FIG. 2, where the specimens were normal bone marrow.

1. Debris and dead cells were removed, each population of cells was gated, and the proportion of each population was shown, as described in steps 1-2 of the AML analysis above.

2. Gating of CD34+ cells: Sequential gating was adopted for CD34/SSC and CD34/CD45 plots to remove non-specific cells. CD34+ cells were gated and CD13 and HLA-DR expression within CD34+ cell gate were analyzed.

3. Gating of CD117+ cells: Sequential gating was adopted for CD117/SSC and CD117/CD45 plots to gate CD117+ cells and remove CD45st+ and non-specific cells. The distribution of CD33/CD13 and CD13/HLADR within CD117+ cells was analyzed. FIG. 2 shows that CD33 versus CD13 plot was in a continuous changing distribution, and CD13 versus HLADR plot was distributed in multi-populations, which was a normal phenotype. Discontinuous or clustered distribution of CD13 versus CD33 plot could occur in MDS, and HLA-DR− cell population increased in CD13 versus HLADR plot, which was abnormal.

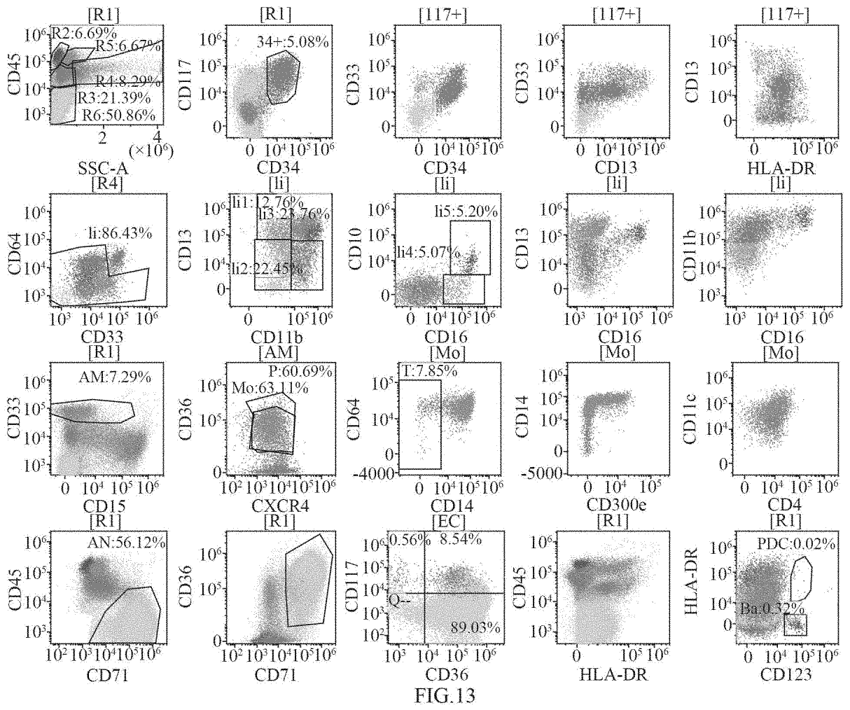

4. Analysis of granulocytes: sequential CD45/SSC, CD33 or CD64 and CD15 gating was adopted for granulocytes with weak CD33 and CD64 expression and strong CD15 expression. Granulocytes were broadly classified into li1 to li5 stages based on CD13/CD11b, CD10/CD16, which roughly corresponded to promyelocytes, myelocytes, metamyelocytes, stab granulocytes, and segmented granulocytes, respectively. Normal bone marrows at li1 and li2 stages were less than 10%. Some MDS showed an increased proportion of li1+1i2 cells. FIG. 2 shows the distribution of the CD13/CD11b, CD13/CD16 and CD11b/CD16 in normal granulocytes, with CD13/CD16 in a checkmark pattern. In some MDS, the distribution was abnormal due to changes in the intensity of CD13, CD11b, and CD16 expression, as shown in FIG. 13.

5. Analysis of monocytes: sequential CD45/SSC, CD33 or CD64 and CD15 gating was used to gate monocytes with strong CD33 and CD64 expression and weak CD15 expression. The analysis was focused on the proportion of promonocytes, in the same way as AML.

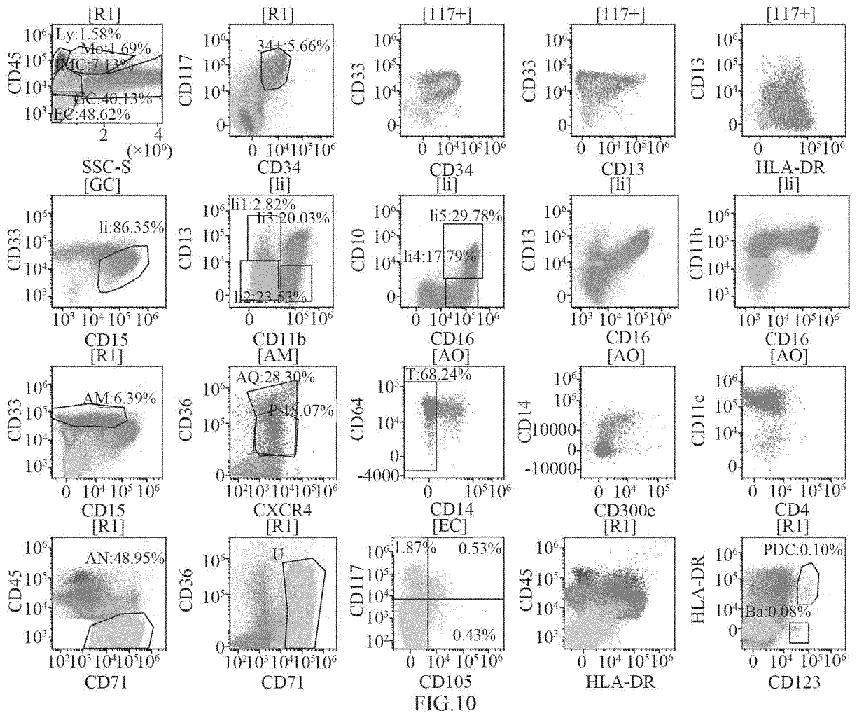

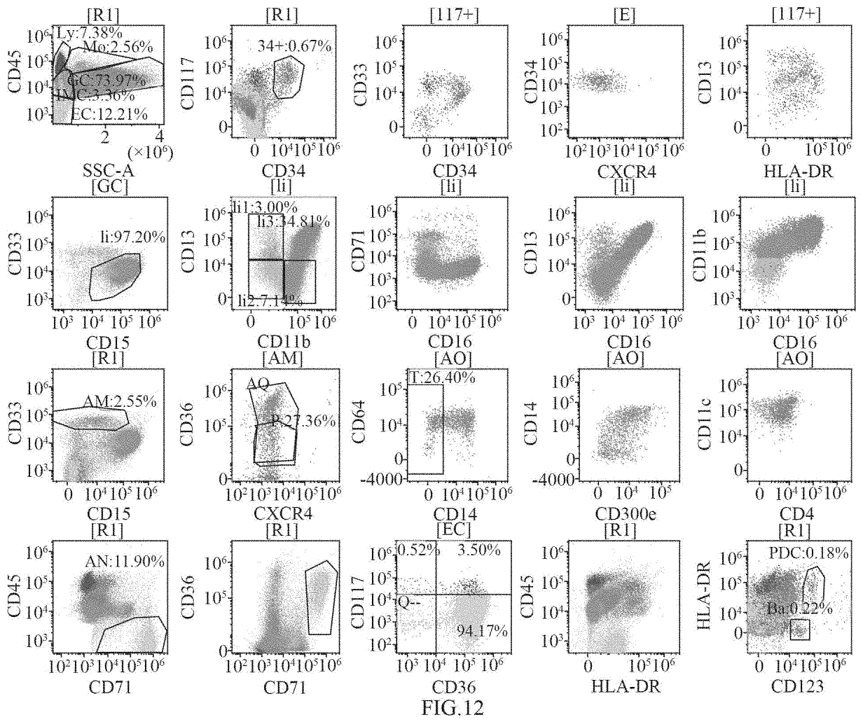

6. Analysis of the phenotype of erythroblasts: sequential CD45/SSC, CD71 or CD36 gating was carried out on CD71+CD45 weakly expressing or negative erythroblasts. The proportion and expression intensity of CD71, CD36, CD105 and CD117 positive cells were observed. Normal phenotype showed strong CD71 and CD36 expression as shown in FIGS. 2 and 12. In MDS (FIG. 10), reduced expression intensity of CD71, CD36, CD105 and a reduced proportion of CD105+ cells (normal >10%) were observed.

7. Analysis of CD123/HLA-DR plot: the proportion of CD123st+ HLA−DR+(pDC) and CD123st+ HLA−DR-basophils (normally <1%) were analyzed.

The diagnosis was then made in conjunction with the results of the primary screening tube as follows: (1) Supported MDS: more than 1% of increase in proportion of immature myeloid cells, or ogata score more than 2, or abnormal immature myeloid phenotype e.g. increased CD34+CD38−%, or CD34st+/dim+ or CD117st+/dim+ or CD33st+/dim+ and CD13st+/dim+ and HLA-DRdim+. (2) Suspected MDS: abnormal granulocytes or erythrocytes only. (3) Excluding MDS: no significant abnormalities in immature myeloid cells, granulocytes or erythrocytes. See Chinese patent application No. 2021110670743 for the method of diagnostic analysis of the primary screening tube.

(iii) Analysis of Chronic Myeloid Neoplasms

The analysis was performed in the same way as for MDS. Kaluz© software was used to remove debris and perform gating analysis on a CD45/SSC plot. Lymphocytes, monocytes, granulocytes, erythroblasts, eosinophils and immature cells were gated and their proportions in the sample were determined. It was determined whether the proportions and the phenotypes of each cell populations were abnormal. The diagnosis was moved on based on the followings in combination with clinical presentation.

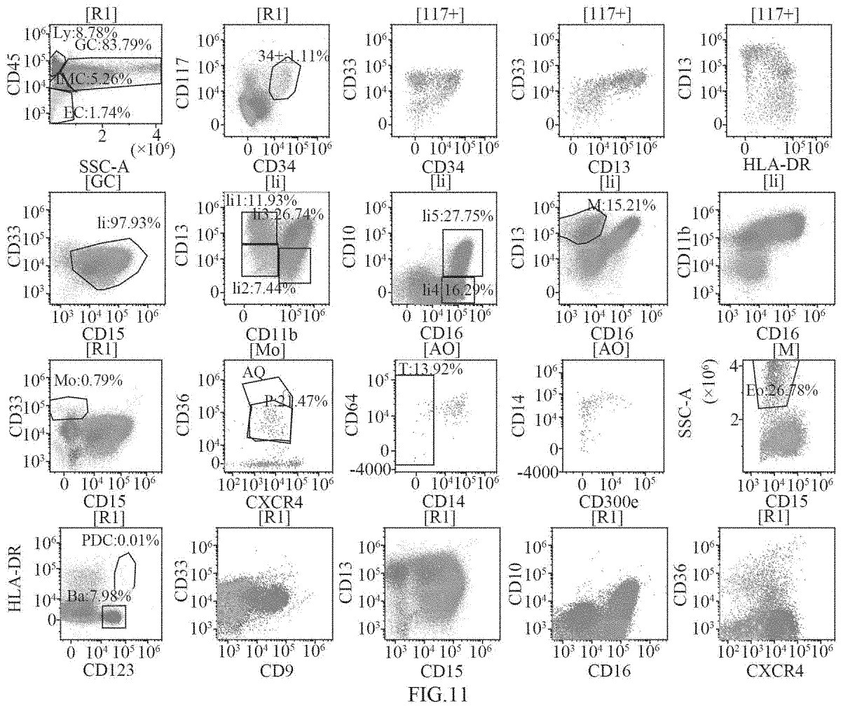

1. Chronic myeloid leukaemia (CML): The manifestations that the proportion of granulocytes is 79-90%, and the percentage of CD34+CD117+ cells is normal or slightly increased, usually below 5%. The proportion of CD11b-granulocytes (li1 and li2 stages, as shown in FIG. 11) in granulocytes is increased by >10% The proportion of CD10+ mature granulocytes is reduced (<40%) and CD15 was weakly expressed. CD10-granulocytes expressing CD56 are increased (>10%) (primary screening tube), and CD123st+ HLA-DR-eosinophils and CD16−CD13+ eosinophils with high SSC intensity are increased (>1% and >5% respectively). Such manifestations, together with a marked increase in white blood cells (WBC) and even splenomegaly, is indicative of CML. Clinical chromosomal and genetic testing is required to confirm the diagnosis.

2. Chronic neutrophilic leukaemia (CNL): No CML phenotype is present. The disease mainly presents with an increased proportion of granulocytes, of which the granulocytes are mainly CD10+CD16+ mature granulocytes, with a normal proportion of CD34+CD117+ and no significant abnormalities in the rest of the cells.

3. MPN-thrombocytosis (essential thrombocythaemia, ET): Patients with ET often manifest a marked increase in platelets, accompanied by a mild increase in WBC. The CD45/SSC and CD34/CD117 plots show no abnormalities in the ratio of each cell populations. The proportion of granulocytes CD11b-immature cells is not high (normally <10%) and there may be a slightly weaker expression of CD11b. The remaining cells are not significantly abnormal. Such manifestation is indicative of MPN-ET.

4. Chronic myelomonocytic leukaemia (CMML) in MDS/MPN: For patients with more than 1×10⁹/L monocytes in peripheral blood, the percentage of monocytes in the bone marrow is mainly analyzed and attention is paid to whether they are mature or promonocytes, identified in the same way as in AML. Predominantly mature monocytes are increased, with >8% monocytes. If CD14, CD300e, CD CD11b, and CD36st are expressed, mature monocytes are identified. Other monocyte related markers may be positive, but HLA-DR, CD11b, CD13 and even CD15 can show abnormal intensity of expression. CD56 is also expressed in some patients. Granulocytes can also show an increased proportion of CD11b-cells. When the percentage of CD34+CD117+ cells are normal or slightly increased and promonocytes are less than 20%, combined with the indication of more than 1-10⁹/L monocytes in the peripheral blood, CMML are diagnosed.

5. Other Chronic Myeloid Neoplasms.

In patients with myelofibrosis (MF), there may be an abnormal immature cell phenotype, e.g. CD38dim+, and other cellular abnormalities may not be evident, and an increase in basophils may be seen.

Analysis of mast cells: The cells are CD117st+, and express CD33 and CD9. In mastocytosis, the antibody panels can be used to detect an increased proportion of CD117 st+ mast cells and analyze whether CD33 and CD9 expression is abnormal.

Analysis of basosphils: HLA-DR-CD123st+ is used to gate eosinophils, which have a small SSC intensity, are weak CD45+ cells, also express CD9st, CXCR4st, CD13, CD33, CD11b, and CD11c and do not express CD15, CD10, CD16, CD64, and CD14. The antibody panels can be used to analyze whether the proportions (normally <1%) and phenotypes are abnormal.

Analysis of eosinophils: Eosinophils have the highest SSC intensity, are positive for CD45st, expresse CD33, CD13, and CD11b, weakly express CD15, and does not express CD16 or CD10. Determination of whether the proportion of eosinophils (normally <5%) and their phenotype is abnormal and whether there are immature eosinophils that can help to determine eosinophilic disease.

IV. Results.

A total of 103 bone marrow specimens were tested using the antibody panels of the present disclosure, all of which underwent primary screening tube testing. Of the specimens, 27 were proved to have AML and were tested using the AML antibody panel. The remaining 76 specimens were tested by primary screening tests. They were excluded from having lymphoid neoplasms or suspected myeloid neoplasms, and thus were tested using the antibody panel for chronic myeloid neoplasms.

It was shown that in 27 cases of AML, the phenotypes after the test were: 12 cases of AML-M2, 10 cases of AML-M4/5 and 1 case of AML-basophilic; 4 cases of AML with recurrent genetic abnormalities: including 1 case of AML with Mutated NMP1, 1 case of AML with t(8:21) and 2 cases of APL with t(15:17). 44 cases were diagnosed with chronic myeloid neoplasms, including: 15 cases of supportive MDS, 5 cases of suspected MDS, 5 cases of MPN, 5 cases of MDS/MPN (including 3 cases of CMML): 6 cases of suspected CML; 4 cases of eosinophilia: and 4 cases of hypoplasia. 32 specimens were approximately normal.

All 103 specimens were simultaneously immunotyped with the conventional 8-10 color 4-8 tube antibody panels and the results were consistent between the 2 methods.

6 cases of CML identified by initial determination were confirmed by genetic testing, and the diagnosis of CML was confirmed in 6 cases with positive CML genes. The above results demonstrate the high sensitivity of the antibody panels of the disclosure for the diagnosis of MDS.

In particular. FIG. 1 shows the role of CXCR4 in the analysis of monocytes. As shown in FIG. 1, A shows the results of an AML-M5 patient using CXCR4/CD36 to identify promonocytes. The CD45/SSC plot shows 5.26% immature (R3) expressing CD117. It also shows 31.93% of R5 expressing CD64, CD14, CD36, CD300e, CXCR4dim, CD11c, and CD4, a significantly higher proportion of mature monocytes. R4 accounted for 42.28%, as shown in FIG. 1A. Cells in R4 gate of the lower row were selected. CXCR4+CD36dim+ cells in the R4 gate were gated as E, with a percentage of 23.33%. Gate E cells expressed CD64 and CD4 but not CD14 and CD300e. CD36 and CD11c expression was weaker than that in normal monocytes, while CXCR4 expression was stronger than that in normal monocytes. Unlike promyelocytes, the gate E cells were located below R5 in the CD45/SSC plot and had a lower SSC intensity. The combined results suggested a promonocyte profile, indicating that R4 included 23.47% promonocytes. The CD45/SSC gating alone could not identify promonocytes. Based on these results in combination with the morphological findings, the final diagnosis was AML-M5.

As shown in FIG. 1B for a case of CMML, the boundary between monocytes, granulocytes and immature cells was seen to be unclear from the CD45/SSC plot, which divided them roughly into granulocytes (R4 gate) and monocytes (R5 gate). However, when R4 gate cells were shown on the CD33/CD15 plot, 3.48% CD33st+CD15dim+ monocytes were observed (top row middle). In contrast, when R5 cells were shown on CD33/CD15 plot, 13.42% of CD33+CD15st+ granulocytes were observed (bottom row left). This indicated that it was not possible to clearly gate granulocytes and monocytes using CD45/SSC. R1 gate cells were selected from the CD64/CD14 plot, and CD64+CD14+/− cells were gated as mono-3, and the mono-3 gate cells were shown in the CXCR4/CD36 plot, which allowed the mono-3 gate cells to be divided into three groups: CD36st+ CXCR4+ mature monocytes, 46.47%; CD36dim+CXCR4+ promonocytes, 26.04%; and CD36-CXCR4−cells, 20.36%. CD36-CXCR4−cells had dimer expression of CD45 and higher SSC intensity in the CD45/SSC plot, and were suggested to be promyelocytes and indicative of CMML. It was shown that this specimen was not well gated using both CD45/SSC and CD64/CD14. In summary, it was shown that the ability to distinguish between promonocytes and promyelocytes using CD36/CXCR4 combined with CD64/CD14, CD14/CD300e and CD11c/CD4 was enhanced, improving the diagnostic ability of immunotyping for CMML and AML-M4/5.

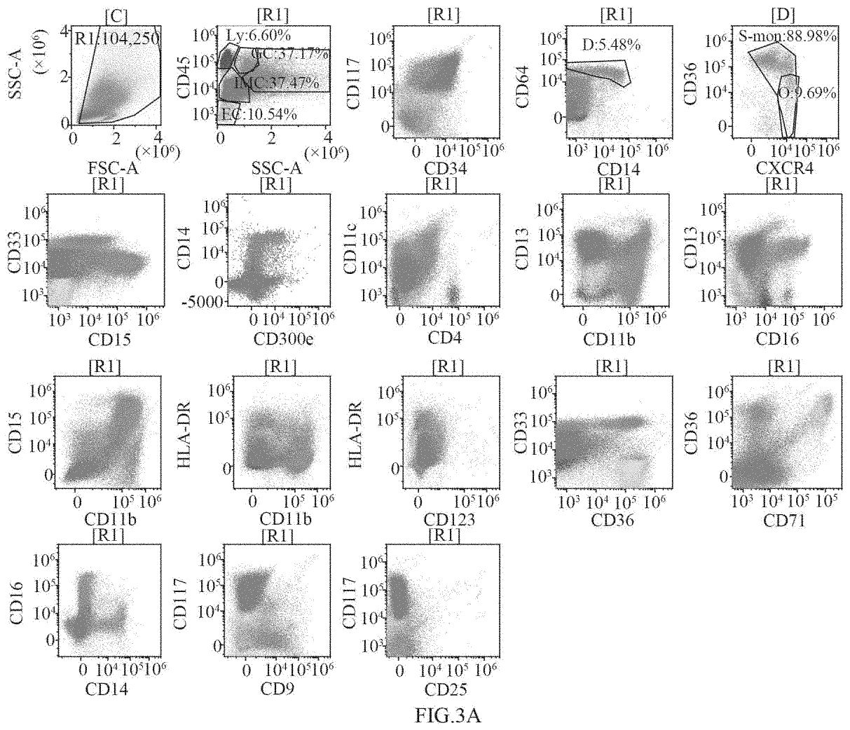

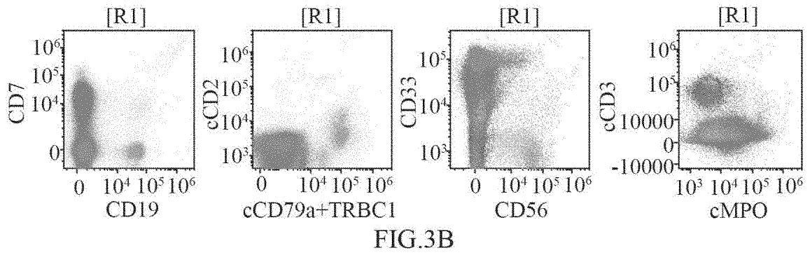

FIG. 3 shows the detection results of an AML-M2 case. The preliminary screening results are shown in FIG. 3B. Further, AML subtyping was performed using the antibody composition of the disclosure, and the results are shown in FIG. 3A. Immature granulocytes accounted for 37.47% as shown in the CD45/SSC plot and expressed CD117, CD34, weak CD33, and CD13, and the rest of the antigens were negative. In the CD64/CD14 plot, CD64st+ cells were subjected to monocyte gating, monocytes accounted for 5.48%, a low percentage. In the CXCR4/CD36 plot, CD36dim+CXCR4+ promonocytes (O gate) accounted for only 9.69% and the rest were mature monocytes, indicating mature monocytes were the most. R4 gate cells accounted for 37.17% and immature granulocytes was more than 20%. Thus, AML-M2 was diagnosed. LAIP: CD34+CD117+ HLA-DR−.

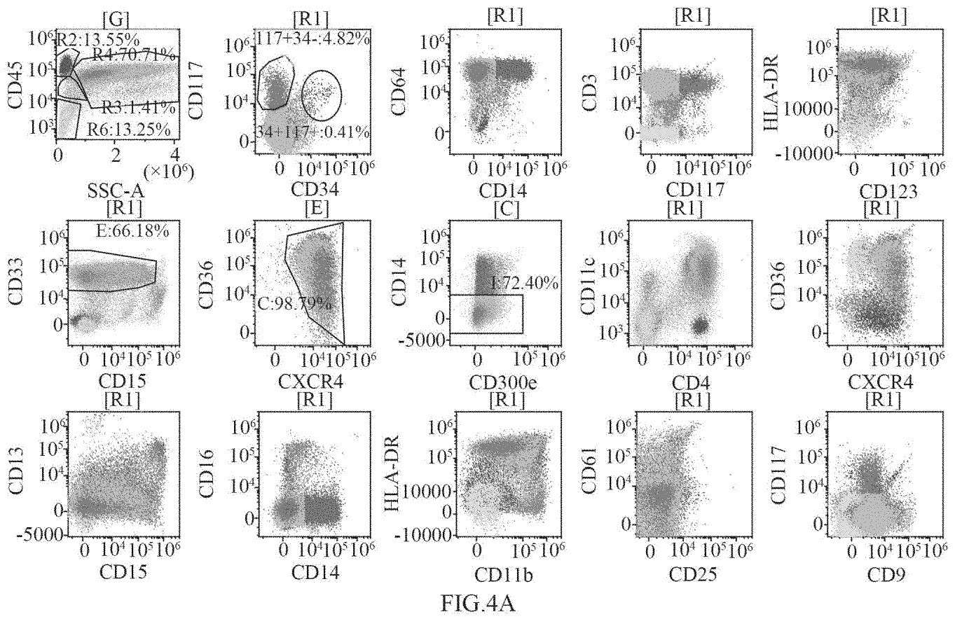

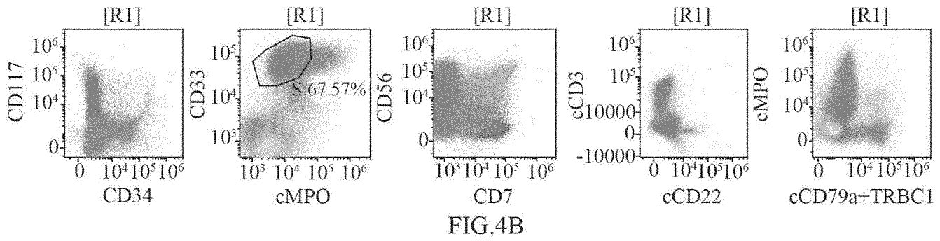

FIG. 4 shows the results of an AML-M5 test in one case. Further, AML subtyping was performed using the antibody combination of the present disclosure, as shown in FIG. 4, A. In the CD45/SSC plot myeloid cells (R4) accounted for 70.71% and it was not possible to determine whether it was monocytes or granulocytes. However, from the CD64/CD14, HLA-DR/CD123 plot, which shows CD64st+CD14part+ and HLA-DR+, it can be judged as monocytes. The CD33/CD15 plot was then used to set gate E for CD33+CD15−, accounting for 66.18% of the cells. From the CD36/CXCR4 and CD14/CD300e plots, this group of cells was seen to be CXCR4+CD36−/+ and CD14part+CD300e−, which can be judged as promonocytes. The simultaneous expression of CD64, CD33, HLA-DR, CD11c, CD4st, partial expression of CD14, CD15 and no expression of CD300e are detected, while CD13 is abnormally negative, are phenotypically abnormal promonocytes, judged as AML-M5. LAIP: CD64+CD33+CD13-HLA-DR+CD56+.

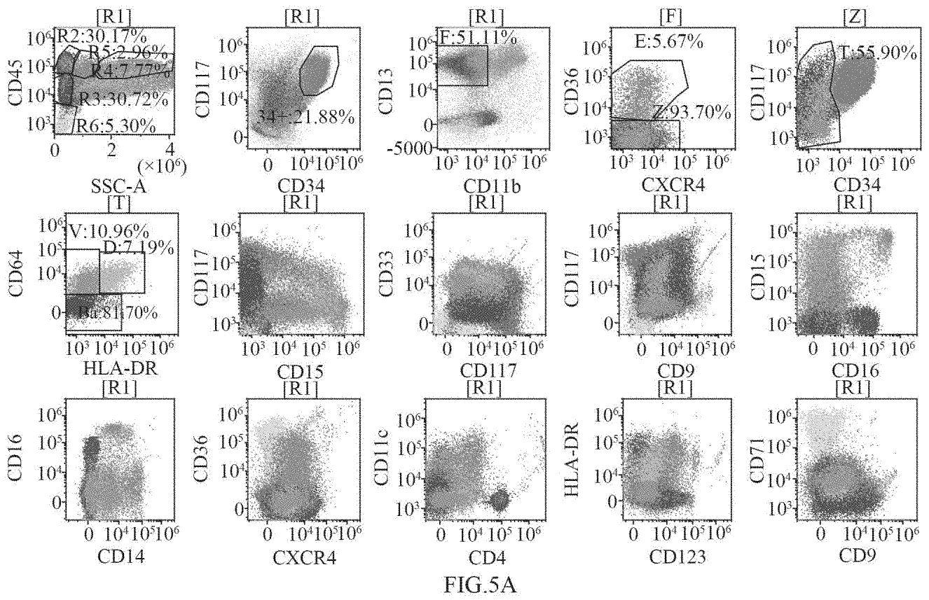

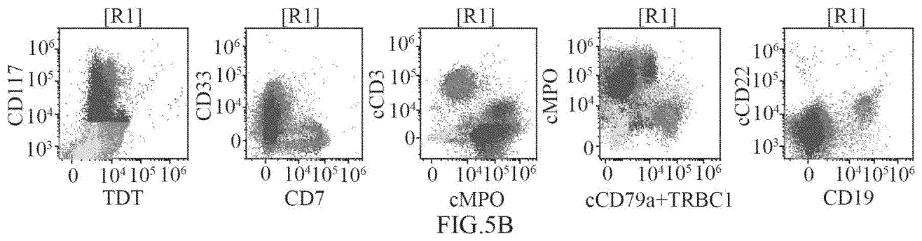

FIG. 5 shows the detection results of a case of AML with basophilic differentiation. The preliminary screening results are shown in FIG. 5A, which indicates the disease is AML, rather than ALL. Further, AML subtyping was performed using the antibody panels of the present disclosure, as shown in FIG. 5 A. The CD45/SSC plot shows 30.17% lymphocytes, 2.96% monocytes (R5), 7.77% granulocytes (R4) and 5.30% erythroblasts (R6). The rest of the cells were not clearly classified. In the CD34/CD117 plot, CD34+ gating was performed and CD34+CD117+ cells accounted for 21.88%. In the CD13/CD11b plot, CD13+CD11b− cells were subjected to F gating to remove CD11b+ mature granulocytes. In CD36/CXCR4 plot, F gate cells were shown, CD36−cells were subjected to Z gating to remove CD36+ monocytes. In the CD117/CD34 plot, Z gate cells were shown and CD34−cells were subjected to T gating to remove CD34+ myeloblasts. In the CD64/HLA-DR plot, T gate cells were shown, and the cells were divided into three clusters: CD64+ HLA-DR− promyelocytes (V gate) at 10.96%; CD64+ HLA-DR+ monocytes (D gate) at 7.19%: CD64-HLA-DR− cells (basophilic gate), at 81.70%. The T gate cells were analyzed within the R1 gate for the expression of other antigens. These cell populations expressed CD13st and CD123, partially CD1I7, CD34, CXCR4, and CD9. In particular, CD33 and CD11b were abnormally negative, CXCR4 and CD9 were abnormally diminished, and the rest of the markers were negative. The cells were identified as abnormal immature basophils, which accounted for 30.93% in R1. LAIP: CD34+CD117+CD9part+ HLA-DR−.

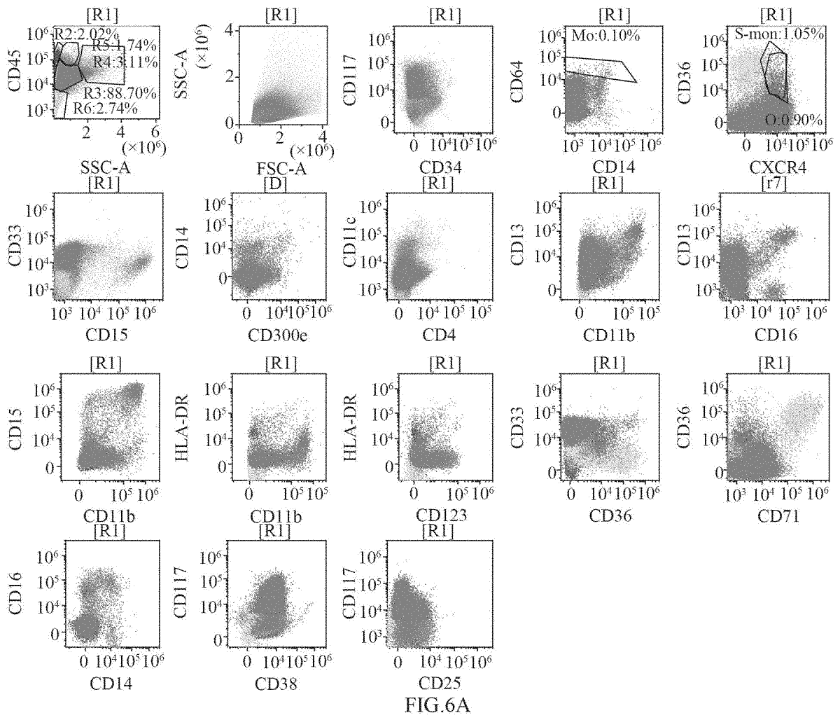

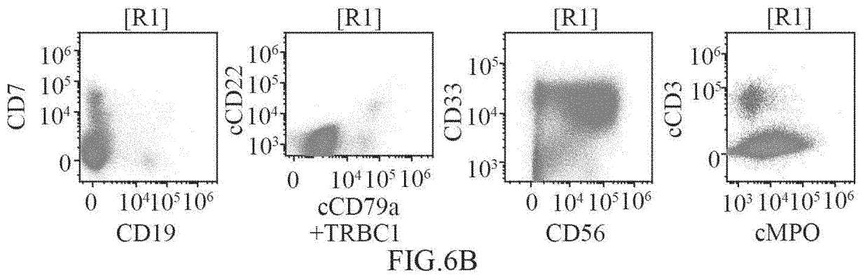

FIG. 6 shows the detection results of a case of AML with mutated NPM1. Preliminary initial screening results are shown in FIG. 6B. It was shown that the cells expressed CD33 and CD56, and were negative for T, B specific markers. Preliminary diagnosis was made with AML, instead of ALL. Further AML subtyping was performed using the antibody panel of the present disclosure. As shown in FIG. 6A, in the CD45/SSC plot, immature granulocytes (R3) accounted for 88.70%, expressed CD117, CD33, and CD38, partially CD13 and CD64, no CD34 and HLA-DR, and were negative for other markers. Decreased CD64st+ monocytes and of granulocytes were observed, each accounting for 0.1% and 3.11%. AML-M2 with mutated NPM1 was diagnosed. LAIP: CD117+CD34-HLA-DR−.

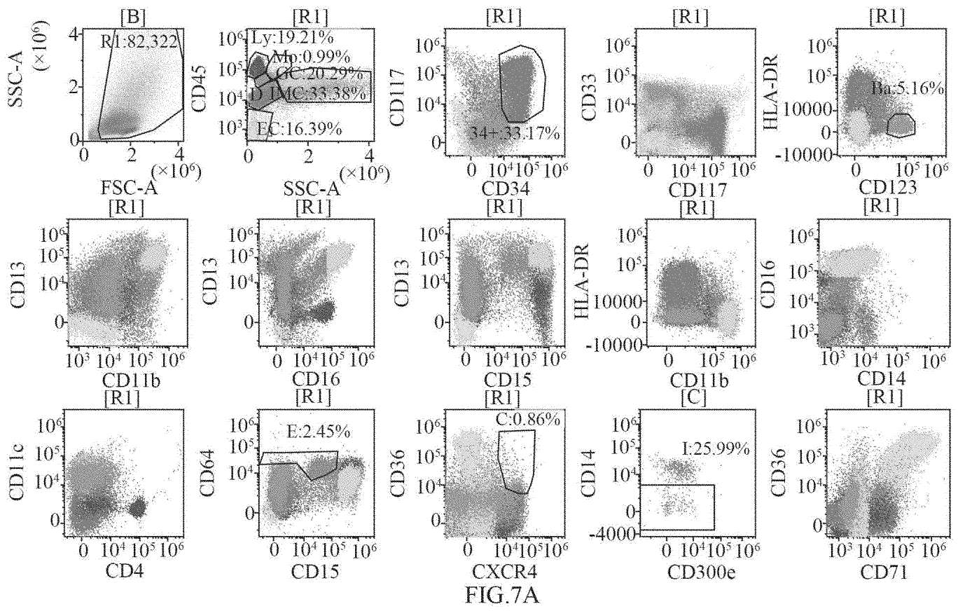

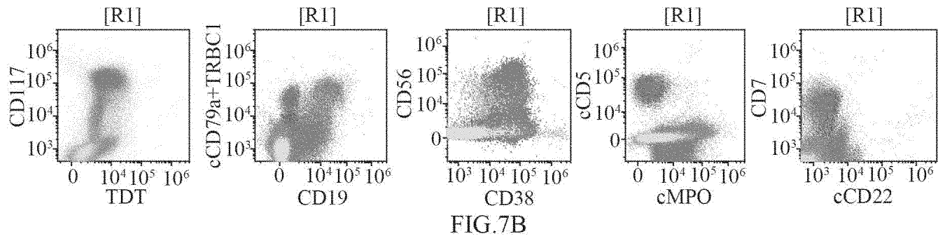

FIG. 7 shows the results of a case of AML with t(8; 21). Preliminary primary screening results are shown in FIG. 7B. The cells were cMPO+, expressed CD33 weakly, partially CD19 and CD56, rarely CD79a, and were negative for CD22 and T-lineage specific markers. The diagnosis was made to be AML with CD19, CD56 expression, rather than ALL. Further AML subtyping was performed using the antibody panel of the disclosure. As shown in FIG. 7A, CD34+CD117+ immature myeloid cells (34+ gate) in the CD34/CD117 plot accounted for 33.17%, expressed CD33dim, CD38, HLA-DR, and CD13, partially CD71 and were negative for the rest of the markers. Monocytes accounted for 2.62%, a small percentage, and were mainly CD64+CD14+CXCXR4+CD36−CD300e− promonocytes with low numbers (percentage <20%). The proportion of granulocytes (R4) was 20.29%, which was reduced. The AML-M2 with t(8; 21) was determined because of the immature granulocyte phenotype of CD33dim+CD19part+CD56part+ and CD34+CD117+CD38+ HLA-DR+. Positivity for RUNX1-RUNX1T1 fusion gene was later confirmed by PCR, so was AML with t(8; 21)(q22; q22), RUNX1-RUNX1T1. LAIP: CD I17+CD34+CD33−CD19part+CD56part+.

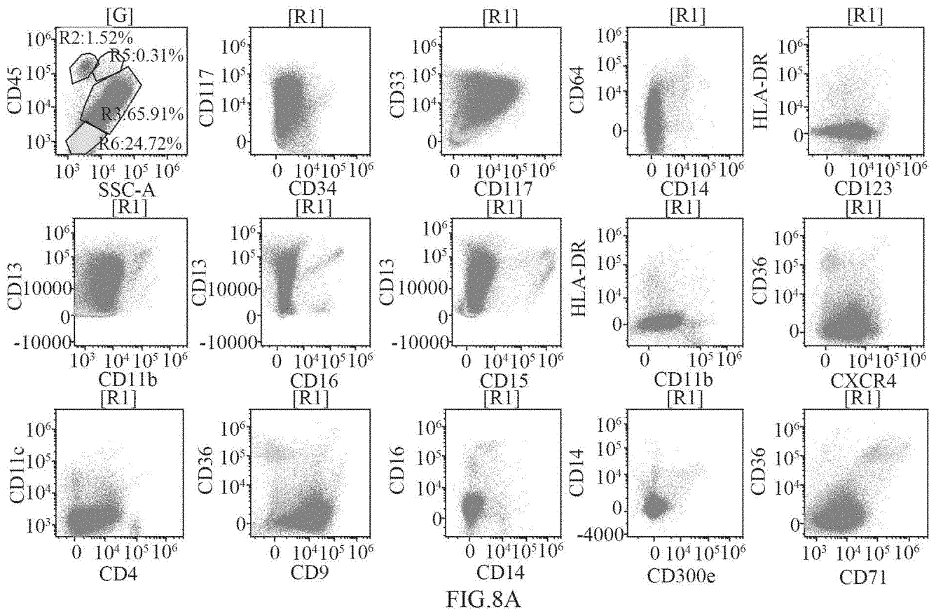

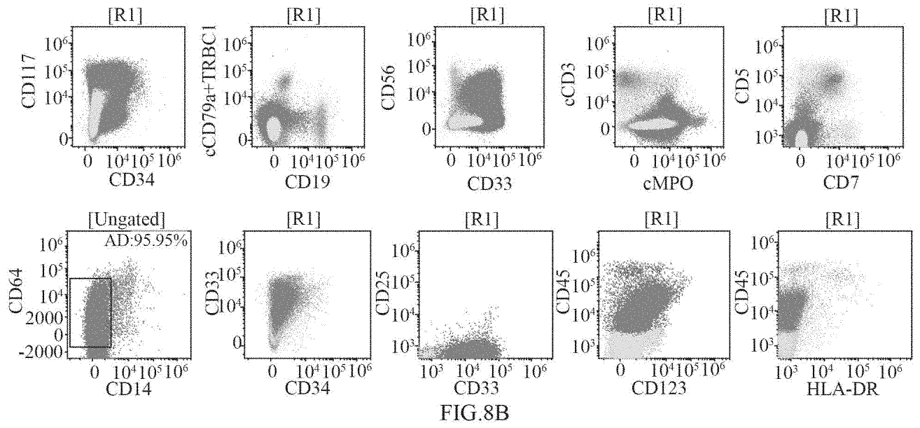

FIG. 8 shows the results of a case of APL with t(15:17). Preliminary primary screening results are shown in FIG. 8B. The cells were cMPO+CD33+cCD3−CD56part+CD5−CD7−CD19−CD79a- and AML, rather than ALL was determined. As shown in FIG. 8A, R3 accounted for 65.91%, had high SSC intensity, expressed CD117, CD33, CD13, and CD9, partially CD64, CD123 CD4, and CXCR4, and no HLA-DR and CD34, and were negative for other markers. There were no obvious monocytes or granulocytes. APL with 1(15:17) was diagnosed because of the CD34-HLA-DR-CD33+CD64dim+CD9+ phenotype and high SSC intensity. Positivity for PML-RARα fusion gene was later confirmed by PCR, so was APL with t(15; 17) (q22; q12), PML-RARα. LAIP: CD117+CD34-HLA-DR-CD33+CD9+CD64dim+ and high SSC intensity.

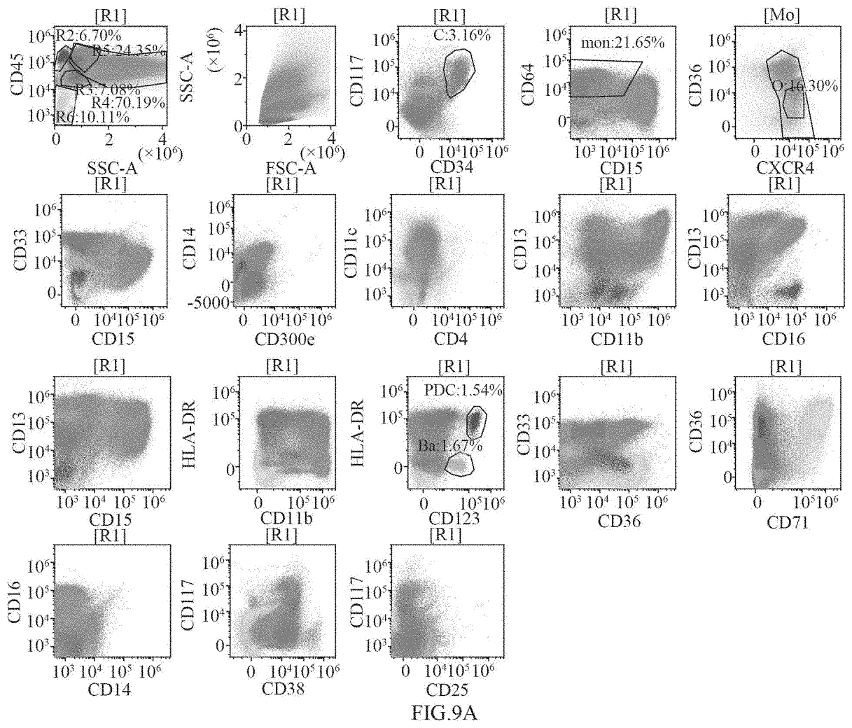

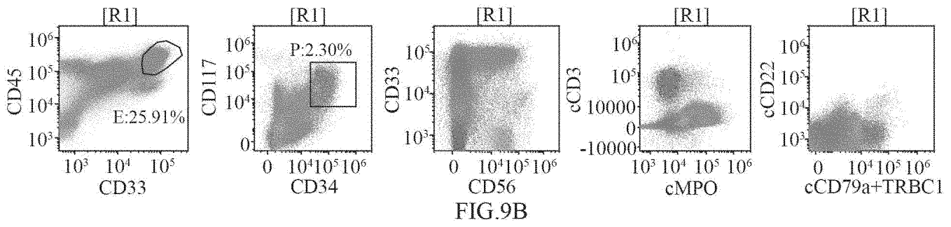

FIG. 9 shows the detection results of a case of CMML. The results of the preliminary primary screening as shown in FIG. 9B showed high monocytes (E) and 2.3% of CD34+CD117+ immature myeloid cells, and CMML were suspected. Further definitive diagnosis was made using the antibody panel of the disclosure, and the results are shown in FIG. 9A, where the CD45/SSC plot showed that the immature cells (R3) were not clearly demarcated from other cell populations. Therefore, CD34+CD117+ immature granulocytes in the CD34/CD117 plot were gated (C), accounting for 3.16%. In the CD64/CD15 plot, CD64st+CD15dim+/− monocytes were gated as mon, accounting for 21.56%, an increased proportion. They strongly expressed CD33, and expressed CD64, CD1c, CD4, CD14, CD300e, CD36, HLA-DR and CD11b. The mon gate cells were shown in a CD36/CXCR4 plot. It was shown that CD36dim-/CXCR4st+ promonocytes (O) accounted for 16.3% and the rest of the monocytes were mature monocytes. CD123st+ HLA-DR− basophils accounted for 1.67%, an increased percentage. This indicated a marked increase in monocytes, predominantly mature monocytes, and diagnosis was made with CMML. LAIP: CD33+CD64+CD56+.

FIG. 10 shows the detection results of a patient with MDS. The CD34/CD117 plot shows 5.66% immature granulocytes (34+) with an increased percentage (normal <1%) and abnormally diminished CD34 expression. CD117+ cells was no longer distributed in a ring shape in the CD33/CD34 plot and appeared as CD33+CD13−, CD13-HLA-DR+ cells with an abnormal phenotype. The proportion of granulocytes within R1 gate reduced to 40.13% and myelocytes at the li2 stage increased to 23.53% (normally, li1+li2<10%). CD33st+CD15dim/−cells were subjected to AM gating and shown in a CD33/CD15 plot, accounting for 6.39%. The AM gate cells were shown in a CD36/CXCR4 plot. It was shown that CD36dim/−CXCR4st+ monocytes (AQ) accounted for 28.3%, a converted low proportion of 1.8% in R1. In particular, most of cells (68.4%) did not express CD14, CD300e and CD4, and were promonocytes, suggesting an abnormal cell differentiation. CD71+CD45−erythroblasts accounted for 48.95%, a significantly increased proportion, with weak expression of CD71 and CD36 and a reduced proportion in CD105+ cells (normal >10%). The proportion of basophils and PDC cells was normal. The diagnosis of MDS was supported. LAIP: CD117+CD34dim+CD13−.

FIG. 11 shows the detection results of a case of CML. The CD45/SSC plot shows a significantly increased proportion of granulocytes (goblet cells) at 83.79%. The CD34/CD117 plot shows a slightly higher percentage of CD34+CD117+ cells (34+ gate) at 1.11%. CD15 was abnormally weakly expressed in granulocytes, with a significantly increased proportion of li1 stage cells at 11.93% (normal li1+1i2<10%). The proportion of CD33st+ monocytes (monocytes gate) was significantly reduced (0.79%). The proportion of CD123st+ HLA-DR-basophils (basophils) was 7.98%, which was significantly higher. From the CD13/CD16 plot, it was observed that a population of CD13+CD16−cells accounted for 15.21% and this population was shown in the SSC/CD15 plot. 26.78% of the cells showed high SSC intensity and weak CD15, and were deemed to be eosinophils with an increased proportion. This patient had a number of WBCs of 182×10⁹/L and was therefore diagnosed to have CML. Genetic testing demonstrated a positive CML gene, P210+.

FIG. 12 shows the results of a case of MPN-ET. The CD45/SSC plot and the CD34/CD117 plot show no abnormalities in the ratio of the cell populations. Promyelocytes at li1 stage and myeolocytes at li2 stage were slightly overrepresented with 3.0% and 7.14% respectively, and CD11b expression was slightly weaker. The rest were not significantly abnormal. These are characteristic of patients with MPN-ET. This disease is not amenable to MRD monitoring by flow cytometry and LAIP was thus not conducted.

FIG. 13 shows the detection results of a case of MDS/MPN UC. The CD34/CD117 plot shows an increased proportion of CD34+CD117+ immature myeloid cells at 5.08%. CD11b in granulocytes was abnormally weakly expressed, the proportion of promyelocytes at li1 stage and myelocytes at li2 stage were significantly higher and the CD13/CD11b and CD13/CD16 plots were significantly abnormal compared to those in FIG. 2. The proportion of monocytes was not high and the phenotype was normal. The proportion of erythroblasts was 50.86%, which was significantly higher. The patient had an increased WBC, anaemia and reduced platelets. MDS/MPN was considered. LAIP: CD34+CD117+CD33dim+.

Although, the disclosure has been described in detail above with a general description and specific examples, some modifications or improvements can be made on the basis of the present disclosure, as will be apparent to those skilled in the art. These modifications or improvements, which do not deviate from the spirit of the disclosure, therefore fall within the protection scope claimed by the present disclosure.

1. A kit for immunotyping of a myeloid neoplasm, comprising a panel of antibodies:
anti-CXCR4 antibody, anti-CD105 antibody, anti-CD14 antibody, anti-CD45 antibody, anti-CD16 antibody, anti-HLA-DR antibody, anti-CD33 antibody, anti-CD10 antibody, anti-CD4 antibody, anti-CD123 antibody, anti-CD11b antibody, anti-CD41 antibody, anti-CD61 antibody, anti-CD15 antibody, anti-CD13 antibody, anti-CD71 antibody, anti-CD117 antibody, anti-CD34 antibody, anti-CD9 antibody, anti-CD11c antibody, anti-CD300e antibody, anti-CD64 antibody, anti-CD36 antibody, and anti-CD25 antibody.

2-3. (canceled)

4. The kit according to claim 1, wherein the antibodies are monoclonal antibodies.

5. (canceled)

6. A system for detecting an immunophenotype of a myeloid neoplasm, comprising a test moiety and an analyzing moiety, wherein
the test moiety is used to attain test results of a sample using a tube of agent for testing the sample with flow cytometry, and the agent comprises the panel of antibodies according to claim 1.

7. The system according to claim 6, wherein when the system is used for detecting an immunophenotype of an AML and/or a chronic myeloid neoplasm, steps for detection comprise:
preparing a flow cytometry onboard sample after processing the sample to be tested using a panel of antibodies; performing a flow cytometry onboard assay;
wherein gating in the flow cytometry on-board assay is carried out as follows:
gating live cells as R1, removing debris and dead cells, and gating lymphocytes, granulocytes, monocytes, immature cells, and erythroblasts within the R1 gate using CD45/SSC; analyzing antigen expression within different cell gates;
analyzing the AML for immunophenotypes of immature myeloid cells, granulocytes and monocytes; and
analyzing the chronic myeloid neoplasm for immunophenotypes of immature myeloid cells, granulocytes, monocytes and erythroblasts;
wherein the panel of antibodies comprises:
anti-CXCR4 antibody, anti-CD105 antibody, anti-CD14 antibody, anti-CD45 antibody, anti-CD16 antibody, anti-HLA-DR antibody, anti-CD33 antibody, anti-CD10 antibody, anti-CD4 antibody, anti-CD123 antibody, anti-CD11b antibody, anti-CD41 antibody, anti-CD61 antibody, anti-CD15 antibody, anti-CD13 antibody, anti-CD71 antibody, anti-CD117 antibody, anti-CD34 antibody, anti-CD9 antibody, anti-CD11c antibody, anti-CD300e antibody, anti-CD64 antibody, anti-CD36 antibody, and anti-CD25 antibody.

8. The system according to claim 7, wherein analysis of immunophenotypes of granulocytes and monocytes comprises identifying immature monocytes from mature monocytes and/or identifying promonocytes from immature granulocytes using CXCR4/CD36 analysis.

9. (canceled)

10. A method for identifying promonocytes, comprising detecting CXCR4 and CD36 antigen expression on cell membranes in a sample to be tested for analysis of promonocytes, wherein the CXCR4 expression is stronger in promonocytes than in mature monocytes and granulocytes, while CD36 is distributed from negative to positive in promonocytes.
